(19) United States
(12) Patent Application Publication
(10) Pub. No.: US 2011/0301199 A1
(43) Pub. Date: Dec. 8, 2011

(54) COMPOSITIONS AND METHODS FOR TREATING MYELODYSPLASTIC SYNDROME

(75) Inventors: Gail L. Brown, Woodside, CA (US); Lixin Meng, Cupertino, CA (US)

(73) Assignee: Telik, Inc.

(21) Appl. No.: 13/108,756

(22) Filed: May 16, 2011

Related U.S. Application Data
(60) Provisional application No. 61/352,374, filed on Jun. 7, 2010.

(51) Int. Cl. A61K 31/454; A61P 7/00

(52) U.S. Cl. 514/323

(57) ABSTRACT

This invention generally relates to compositions and methods for treating myelodysplastic syndrome. In one embodiment, this invention relates to methods for treating myelodysplastic syndrome with ezatiostat or a salt thereof in patients who have been treated with a DNA methyltransferase inhibitor by a combination of ezatiostat or a salt thereof and lenalidomide.

### CROSS-REFERENCE TO RELATED APPLICATION

This application claims the benefit under 35 USC 119(e) of U.S. Provisional Application No. 61/352,374, filed Jun. 7, 2010, which is incorporated herein by reference in its entirety.

### FIELD OF THE INVENTION

This invention relates to compositions and methods for treating myelodysplastic syndrome.

### STATE OF THE ART

Myelodysplastic syndrome(s) (MDS) refers to a heterogeneous group of clonal hematopoietic stem cell disorders characterized by ineffective hematopoiesis (blood cell production) involving one or more cell lineages (red blood cells, white blood cells or platelets) and a variable risk of transformation to acute myeloid leukemia (AML). This syndrome becomes more common with age. It is estimated that MDS affects approximately 300,000 people worldwide. According to the American Cancer Society, 10,000 to 20,000 new cases of MDS are diagnosed each year in the United States alone. Survival rates using current therapy range from 6 months to 6 years with patients often requiring blood transfusions to manage their disease.

Currently, there are three approved drugs for treating MDS by the U.S. Food and Drug Administration (FDA). Lenalidomide is indicated for the treatment of transfusion dependent MDS patients with del(5 q) and lower risk disease while azacytidine and decitabine are approved for all categories. With the exception of del(5 q) patients, the response rate is approximately 50%, highlighting the need for clinical trials of new agents.

Ezatiostat and its salts are disclosed in U.S. Pat. No. 5,763,570. Ezatiostat has the IUPAC chemical name of ethyl (2S)-2-amino-5-[[(2R)-3-benzylsulfanyl-1-[[(1R)-2-ethoxy-2-oxo-1-phenylethyl]amino]-1-oxopropan-2-yl]amino]-5-oxopentanoate.

One example of a salt of ezatiostat is the hydrochloride salt, ezatiostat hydrochloride (USAN), which has the molecular weight of 566.1, the trademark of Telintra®, and the CAS registry number of 286942-97-0. U.S. patent application No. 13/041,136, filed Mar. 4, 2011, describes ansolvate and polymorphs of ezatiostat hydrochloride.

Ezatiostat hydrochloride has been evaluated for the treatment of MDS, in a Phase I-IIa study using a liposomal formulation (U.S. Pat. No. 7,029,695), as reported at the 2005 Annual Meeting of the American Society for Hematology (Abstract #2250) and by Raza et al. in Journal of Hematology & Oncology, 2:20 (published online on 13 May 2009); and in a Phase I study using a tablet formulation, as reported at the 2007 Annual Meeting of the American Society for Hematology (Abstract #1454) and by Raza et al. in Blood, 113:6533-6540 (prepublished online on 27 Apr. 2009), and in a single patient case report by Quddus et al. in Journal of Hematology & Oncology, 3:16 (published online on 23 Apr. 2010).

The entire disclosures of each of the patents, patent applications, and publications referred to in this application are incorporated into this application by reference.

### SUMMARY OF THE INVENTION

This invention relates to the discovery of the problem that patients with a myelodysplastic syndrome who have been treated with a DNA methyltransferase inhibitor did not respond to treatment with ezatiostat hydrochloride. This invention is further based on the surprising discovery that the response rate to ezatiostat hydrochloride is increased in patients who had lenalidomide treatment prior to administration of ezatiostat hydrochloride.

Accordingly, in one aspect, this invention is directed to a method for treating a myelodysplastic syndrome in a patient who has been treated with a DNA methyltransferase inhibitor, which method comprises administering to said patient lenalidomide prior to and/or concurrently with administration of ezatiostat or a salt thereof.

In some embodiments, ezatiostat or a salt thereof is administered by a dosing regimen described in U.S. patent application Ser. No. 13/______, titled “COMPOSITIONS AND METHODS FOR TREATING MYELODYSPLASTIC SYNDROME,” filed on even date under the Attorney Docket No. 056274-4301, which is incorporated by reference in its entirety and claims priority to U.S. Provisional Application No. 61/352,371, filed on Jun. 7, 2010. For example, ezatiostat or a salt thereof may be administered in cycles of 2 gram/day orally for 3 weeks on/1 week off, or cycles of 3 gram/day orally for 2 weeks on/1 week off. Equivalent ezatiostat doses for ezatiostat itself or other ezatiostat salts, or for other routes of administration may also be used.

In one embodiment, ezatiostat or a salt thereof can be administered as a tablet formulation. Such a tablet formulation is disclosed in U.S. patent application No. 13/075,116, filed Mar. 29, 2011, titled “TABLET FORMULATION OF EZATIOSTAT,” which is incorporated by reference in its entirety.

In another aspect, this invention provides a composition for treating a myelodysplastic syndrome in a patient who has been treated with a DNA methyltransferase inhibitor, which composition comprises lenalidomide, ezatiostat or a salt thereof, and optionally a pharmaceutically acceptable excipient.

In still another aspect, this invention provides a kit of parts for treating a myelodysplastic syndrome in a patient who has been treated with a DNA methyltransferase inhibitor, which kit comprises a first composition comprising lenalidomide and a second composition comprising ezatiostat or a salt thereof.

These and other embodiments of this invention are further described in the text that follows.

### DETAILED DESCRIPTION OF THE INVENTION

Prior to describing this invention in greater detail, the following terms will first be defined.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to “a DNA methyltransferase inhibitor” includes a plurality of DNA methyltransferase inhibitors (DMTIs).

### 1. DEFINITIONS

Unless defined otherwise, all technical and scientific terms used herein have the same meaning as commonly understood by one of ordinary skill in the art to which this invention belongs. As used herein the following terms have the following meanings.

The term “comprising” or “comprises” means that the compositions and methods include the recited elements, but do not exclude others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” means excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 15%, 10%, 5% or 1%.

“Lenalidomide” (Revlimid®, also known as Revamid in the UK) is an immunomodulatory agent with antiangiogenic and antineoplastic properties. It has the chemical name of 3-(4-amino-1-oxoisoindolin-2-yl)piperidine-2,6-dione or 1-oxo-2-(2,6-dioxopiperidin-3-yl)-4-aminoisoindoline or 3-(7-amino-3-oxo-1H-isoindol-2-yl)piperidine-2,6-dione, and CAS registry number of 191732-72-6. Lenalidomide is indicated for the treatment of patients with transfusion-dependent due to low- or intermediate-1 risk MDS associated with a deletion 5q cytogenetic abnormality. Lenalidomide is available in 5 milligram (mg), 10 mg, 15 mg and 25 mg capsules for oral administration.

The term “therapeutically effective amount” refers to the amount of either lenalidomide or ezatiostat or a salt thereof that is an amount sufficient to effect treatment, as defined herein, when administered to a subject in need of such treatment. In one embodiment, the therapeutically effective amount will be up to 3.5 grams (g) of ezatiostat or a salt thereof administered per day. Preferably, ezatiostat or a salt thereof is administered in an amount of 2 grams per day and, more preferably, is administered twice a day in equal 1 gram doses. Such a therapeutically effective amount is particularly relevant when the treatment regimen is for 3 weeks of administration of ezatiostat or a salt thereof followed by a week of no administration of the drug. In another embodiment, the therapeutically effective amount will be up to 3 grams of ezatiostat or a salt thereof administered in a single dose, or in 2 equal daily doses of up to 1.5 grams. Such a therapeutically effective amount is particularly relevant when the treatment regimen is for 2 weeks of administration of ezatiostat or a salt thereof followed by a week of no administration of the drug. Preferably, the dosing regimen employs 2 grams of ezatiostat or a salt thereof administered in an amount of 1 gram doses twice a day either under continuous administration or with administration for 3 weeks followed by a week of no administration of the drug.

In a preferred embodiment, the therapeutically effective amount will provide efficacious results in at least about 10% of the treated population, and preferably at least about 15%.

As used herein, the term “treatment” or “treating” means any treatment of MDS in a patient which produces one or more of the following:
inhibiting the MDS, that is, arresting or suppressing the development of symptoms (e.g., need for blood transfusion, abnormal blood count, and the like); and/or
relieving the MDS, that is, causing the regression of symptoms.

As used herein, the term “patient” refers to mammals and includes humans and non-human mammals.

### 2. METHODS

In one aspect, this invention is directed to a method for treating a myelodysplastic syndrome (MDS) in an MDS patient who has been treated with a DNA methyltransferase inhibitor (DMTI).

DMTIs, also known as demethylating agents, are a class of agents that inhibit methylation of DNA through inhibition of the DNA methyltransferase activity. Methylation of DNA is a major mechanism that regulates gene expression in cells. When there is an increase in DNA methylation this can result in the blockage of the activity of “suppressor genes” that regulate cell division and growth.

Examples of DMTIs include analogs of the nucleoside deoxycitidine, such as azacitidine (5-azacytidine), decitabine (5-aza-2′-deoxycytidine), 1-β-d-arabinofuranosyl-5-azacytosine and dihydro-5-azacytidine; and antisense oligodeoxynucleotide, such as MG98 (by MGI Pharma, Inc.), which is directed against the 3′-untranslated region of the DNA methyltransferase-1 enzyme mRNA and is now under clinical study. Other DMTIs are described in Lyko F and Brown R., J. Natl. Cancer Inst., 2005; 97(20):1498-506, which is incorporated hereby by reference in its entirety.

5-Azacytidine (or azacitidine (INN), Vidaza®, CAS registry number 320-67-2), is an analogue of cytidine and has the formula:

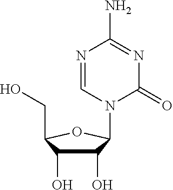

Decitabine (or 5-aza-2′-deoxycytidine, Dacogen®, CAS registry number of 2353-33-5), is a cytosine nucleoside (cytidine) analog and the deoxy derivative of azacitidine, which has the formula:

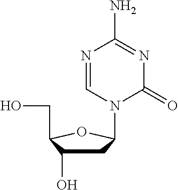

Both azacitidine and decitabine are used in the treatment of myelodysplastic syndrome.

Zebularine (CAS registry number of 3690-10-6) is another DMTI, which has the formula:

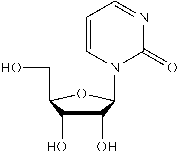

DMTIs cause many side effects, including, but are not limited to: low blood counts (where white, red blood cells and platelets may temporarily decrease, which may put the patient at increased risk for infection, anemia and/or bleeding, and may increase the need for blood or platelet transfusions), fatigue, fever, nausea, cough, petechiae (which can occur with low platelet count), constipation, diarrhea, hyperglycemia, headache, difficulty sleeping, swelling, low albumin, low magnesium, chills, low potassium, bruising, rash, low sodium, dizziness, generalized aches and pains, cardiac murmur, poor appetite, sore throat, abdominal pain, high bilirubin blood level, high potassium, mouth sores, drowsiness, abnormal liver function blood tests, confusion, anxiety, itching, and heartburn.

Due to these side effects, some patients who are on a DMTI would prefer to switch to another therapy, some of these patients cannot continue with the DMTI therapy and must switch to another agent for treating the MDS. It is also desirable for patients who do not respond or respond unsatisfactorily to a DMTI to switch to another MDS therapy. Other MDS patients who have been administered a DMTI for treating a tumor, may need an MDS therapy other than a DMTI. However, it has been unexpectedly found in a clinical trial that ezatiostat hydrochloride, a potential MDS agent, did not exhibit efficacy in patients who had prior exposure to DMTI treatment. As shown in Table 2, none of the evaluable patients in that study who had exposure to at least one DMTI prior to administration of ezatiostat hydrochloride responded to ezatiostat hydrochloride, whereas the response rate to ezatiostat hydrochloride in patients with no prior exposure to DMTI treatment is about 22%. Such results limit the patient's choice in selecting an alternative therapy to replace the DMTI.

This unexpected problem can be solved by the surprising discovery that administration of lenalidomide prior to and/or currently with administration of ezatiostat hydrochloride to MDS patients who has prior exposure to a DMTI can retain the therapeutic effect of ezatiostat hydrochloride in treating an MDS. As shown in Table 2, the response rate to ezatiostat hydrochloride recovered to about 20% in patients who were treated with both lenalidomide and a DMTI prior to administration of ezatiostat hydrochloride.

Accordingly, in one aspect, this invention is directed to a method for treating a myelodysplastic syndrome in a patient in need thereof who has been treated with a DNA methyltransferase inhibitor, which method comprises administering to said patient an amount of lenalidomide prior to and/or currently with administration of a therapeutically effective amount of ezatiostat or a pharmaceutically acceptable salt thereof.

In some embodiments, the patient has been treated with at least one dosage of a DMTI. In some embodiments, the patient has been treated with the DMTI for at least 2 days, 3 days, 4 days, 5 days, or 6 days. In some embodiments, the patient has been treated with the DMTI for at least one week, two weeks or three weeks. In some embodiments, the patient has completed at least 1, 2, 3, 4, 5, or 6 treatment cycles. In some embodiments, the DMTI treatment is immediately prior to the administration of ezatiostat or a salt thereof. As used herein “immediately” means that the last DMTI dosage is administered no more than about one day prior to the first administration of ezatiostat or a salt thereof. In some embodiments, the DMTI treatment is less than 1 week prior to the administration of ezatiostat or a salt thereof. In some embodiments, the DMTI treatment is less than 1 month prior to the administration of ezatiostat or a salt thereof. In some embodiments, the DMTI treatment is less than 2 months, 6 months, or 12 months prior to the administration of ezatiostat or a salt thereof.

In some embodiments, the patient needs concurrent treatment with DMTI and ezatiostat or a salt thereof.

In some embodiments, lenalidomide is administered prior to administration of ezatiostat or a salt thereof. A typical lenalidomide treatment schedule involves a 28-day-cycle, during which lenalidomide is administered once a day every day for 21 days (3 weeks) followed by an interruption of 7 days (1 week) when no lenalidomide is administered. This 28-day-cycle can be repeated for a duration of up to 6 months. Lenalidomide capsules have four different strengths: 5 mg, 10 mg, 15 mg, and 25 mg.

In some embodiments, the patient is treated with at least one dosage of lenalidomide prior to administration of ezatiostat or a salt thereof. In some embodiments, the patient is treated with lenalidomide for at least 2 days, 3 days, 4 days, 5 days, or 6 days prior to administration of ezatiostat or a salt thereof. In some embodiments, the patient is treated with lenalidomide for at least one week, two weeks or three weeks prior to administration of ezatiostat or a salt thereof. In some embodiments, the patient is treated with lenalidomide for 1, 2, 3, 4, 5, or 6 treatment cycles prior to administration of ezatiostat or a salt thereof. In some embodiments, the patient is treated with the entire 6-month lenalidomide treatment regimen prior to administration of ezatiostat or a salt thereof.

In some embodiments, the patient is concurrently administered lenalidomide and ezatiostat or a salt thereof. In these cases, the patient may or may not be treated with lenalidomide prior to administration of ezatiostat or a salt thereof. When the patient is on prior lenalidomide treatment, the lenalidomide treatment may continue with administration of ezatiostat or a salt thereof at the same dosage and/or frequency, or at a reduced dosage and/or frequency, or treatment with lenalidomide may completely stop.

When administered concurrently, lenalidomide and ezatiostat or a salt thereof can be administered in any manner in which the pharmacological effects of both are manifested in the patient at the same time. Thus, concurrent administration of lenalidomide and ezatiostat or a salt thereof does not require that a single pharmaceutical composition, the same dosage form, or the same route of administration be used for the two agents. The two agents do not need to be administered at the same time or for a similar length of time. When administered by the same dosage form and the same route of administration, at substantially the same time, it could proceed by delivering both active ingredients simultaneously in a single novel pharmaceutical composition in accordance with the present invention. It is understood that in addition to the above, this invention contemplates that a concurrent administration may be the administration of a first and second pharmaceutical composition comprising lenalidomide and ezatiostat or a salt thereof, respectively. The term “concurrent” includes both simultaneous delivery as well as sequential delivery wherein each drug is administered separately in a manner that provides serum levels of both drugs in the patient at the same time.

In some embodiments of this invention, when administered concurrently with ezatiostat or a salt thereof, lenalidomide is administered in the typical 28-day-cycle as described above and may be given in any of the dosage strengths. In some embodiments, lenalidomide is administered at a reduced dosage and frequency, for example, lenalidomide may be administered once every other day, once every 3, 4, 5, or 6 days. Or it may be administered once a week or may be discontinued while treatment with ezatiostat or a salt thereof continues.

In some embodiments, the patient's prior exposure to DMTI is before administration of lenalidomide to the patient. In some embodiments, the patient's prior exposure to DMTI is after administration of lenalidomide to the patient. In some embodiments, the patient's prior exposure to DMTI is concurrent with administration of lenalidomide to the patient.

In some embodiments of this invention, ezatiostat or a salt thereof, for example, ezatiostat hydrochloride, is administered by a dosing regimen described in U.S. patent Application No. 13, titled “COMPOSITIONS AND METHODS FOR TREATING MYELODYSPLASTIC SYNDROME,” filed on even date under the Attorney Docket No. 056274-4301, which is incorporated by reference in its entirety.

Typically, ezatiostat or a salt thereof is administered in a therapeutically effective amount. In some embodiments of this invention, ezatiostat or a salt thereof is administered up to about 3.5 grams per day of ezatiostat hydrochloride, or an equivalent amount (in terms of ezatiostat content) of ezatiostat itself or another salt of ezatiostat. In a preferred embodiment, the dosing of ezatiostat or a salt thereof is a therapeutically effective amount of up to about 1.5 grams administered twice a day (b.i.d.).

In some embodiments, ezatiostat or a salt thereof is administered daily for at least 2 weeks. In some embodiments, ezatiostat or a salt thereof is administered daily for at least 3 weeks.

In one embodiment of this invention, ezatiostat or a salt thereof is administered in 1 gram dosages twice a day for three weeks followed by an interruption of one week where ezatiostat or a salt thereof is not administered. After the interruption, the regimen can be repeated as necessary. This regimen may be referred to as the “three-week regimen.”

In another embodiment of this invention, ezatiostat or a salt thereof is administered in 1.5 gram dosages twice a day for two weeks followed by an interruption of one week where ezatiostat or a salt thereof is not administered. After the interruption, the regimen can be repeated as necessary. This regimen may be referred to as the “two-week regimen.”

In another embodiment of this invention, the patient is treated continuously with a therapeutically effective amount of ezatiostat or a salt thereof of up to 3 grams per day preferably administered in up to 1.5 gram dosages twice a day. In this embodiment, ezatiostat or a salt thereof can be administered so long as the patient is in need of and can tolerate such treatment. It is contemplated that in this embodiment, the therapeutically effective amount of ezatiostat or a salt thereof may be less or more than that when there is an interruption in the treatment regimen. This regimen may be referred to as the “continuous regimen.”

While twice a day administration is preferred, it is contemplated that once a day administration or 3 times a day administration could be employed. In the former case, once a day administration would assist in patient compliance; whereas in the latter case, smaller tablets could be used for those patients who have difficulty swallowing larger tablets. The amount of drug administered would be adjusted so that the total drug administered per day is a therapeutically effective amount.

The treatment with ezatiostat or a salt thereof may involve one or a combination of two or more of the dosing regimens described herein. The following are exemplifying dosing schedules of ezatiostat hydrochloride:
1.5 gram ezatiostat hydrochloride administered twice per day for 2 weeks for an aggregate total dosing of 42 grams followed by a week when no ezatiostat or a salt g is administered;
1 gram ezatiostat hydrochloride administered twice per day for 3 weeks for an aggregate total dosing of 42 grams followed by a week when no ezatiostat or a salt is administered;
1 gram ezatiostat hydrochloride administered twice per day continuously until the attending clinician deems it appropriate for the patient to be withdrawn from administration;
a therapeutically effective amount of up to 3 grams of ezatiostat hydrochloride per day administered in one, two, or three divided doses for 2 weeks followed by a week when no ezatiostat or a salt is administered;
a therapeutically effective amount of up to 2 grams of ezatiostat hydrochloride per day administered in one, two, or three divided doses for 3 weeks followed by a week when no ezatiostat or a salt is administered; and/or
a therapeutically effective amount of up to 2 grams of ezatiostat hydrochloride per day administered in one, two, or three divided doses continuously until the attending clinician deems it appropriate for the patient to be withdrawn from administration.

An equivalent amount of ezatiostat or another salt thereof (in terms of ezatiostat content) may replace ezatiostat hydrochloride in the above dosings.

When administration of ezatiostat or a salt thereof is twice a day, it is preferred that the interval between the first and second doses be from about 6 to 14 hours and preferably between about 8 and 14 hours.

In one embodiment, ezatiostat or a salt thereof, e.g., ezatiostat hydrochloride, can be administered intravenously as a lipid formulation such as those described in U.S. Pat. No. 7,029,695 which is incorporated by reference in its entirety.

In one embodiment, ezatiostat or a salt thereof can be administered orally. In another embodiment, ezatiostat or a salt thereof can be administered as a tablet formulation. Such a tablet formulation is disclosed in U.S. patent application No. 13/075,116, filed Mar. 29, 2011, titled “TABLET FORMULATION OF EZATIOSTAT,” which is incorporated by reference in its entirety.

### 3. COMPOSITIONS

In another aspect, this invention provides a composition for treating a myelodysplastic syndrome in a patient who has been treated with a DNA methyltransferase inhibitor, which composition comprises lenalidomide, ezatiostat or a salt thereof, and optionally a pharmaceutically acceptable excipient. In some embodiments, the ezatiostat or the salt thereof and the lenalidomide together are in a therapeutically effective amount.

In some embodiments, the composition comprises about 5 mg, 10 mg, 15 mg or 25 mg of lenalidomide and about 200 mg, 300 mg, 400 mg, 500 mg, 600 mg, 700 mg, 800 mg, 900 mg, or 1000 mg ezatiostat or a salt thereof.

In one embodiment, lenalidomide may be added to a tablet formulation of ezatiostat or a salt thereof. Such a tablet formulation is disclosed in U.S. patent application No. 13/075,116, filed Mar. 29, 2011, titled “TABLET FORMULATION OF EZATIOSTAT,” which is incorporated by reference in its entirety.

### 4. KIT

In still another aspect, this invention provides a kit for the treatment of MDS in a patient who has been treated with a DNA methyltransferase inhibitor, which kit comprises a first composition comprising lenalidomide and a second composition comprising ezatiostat or a salt thereof, including those described herein. In some embodiments, the ezatiostat or the salt thereof and the lenalidomide together are in a therapeutically effective amount.

In some embodiments, the kit further comprises a label with instructions to administer the first dose of lenalidomide 1 day, 2 days, 3 days, 4 days, 5 days, 6 days before the first administration of ezatiostat or a salt thereof. In some embodiments, the kit further comprises a label with instructions to administer lenalidomide 1 week, 2 weeks, 3 weeks, 4 weeks, 5 weeks, or 6 weeks before administration of ezatiostat or a salt thereof. In some embodiments, the kit further comprises a label with instructions to administer lenalidomide concurrently with ezatiostat or a salt thereof. In some embodiments, the kit further comprises a label with instructions to administer lenalidomide and ezatiostat or a salt thereof according to any of the dosing schedules described herein.

### EXAMPLES

The present invention is further defined by reference to the following examples. It will be apparent to those skilled in the art that many modifications, both to materials and methods, may be practiced without departing from the scope of the current invention.

### Ezatiostat Hydrochloride Tablets in Patients with an International Prognostic System Score (IPSS) Low to Intermediate-1 Risk Myelodysplastic Syndrome

Eighty-seven patients were randomized and treated at 23 investigational sites. After initial dose ranging in 14 patients, two dose levels were selected for further study. Subsequently, 37 patients were treated at 3 grams daily for two weeks followed by a one week rest period, and 36 patients were treated at 2 grams daily for three weeks followed by a one week rest period. The data on these 73 patients was pooled for this preliminary analysis.

The median age was 72 years, with a patient population distribution of IPSS low risk (23 patients, 32%) and intermediate-1 risk (50 patients, 68%). Patients had received a median of three prior MDS therapies including, 34 patients (47%) with prior Revlimid® (lenalidomide) and 28 patients (38%) with prior DNA methyltransferase inhibitors (DMTI) [azacitidine, decitabine].

At the time of preliminary analysis, 8 patients remained on treatment for continuing clinical benefit. The overall Hematologic Improvement-Erythroid (HI-E) rate was 22%, 13 of 60 evaluable patients (95% CI, 12.1-34.2). The median duration of HI-E response was 46 weeks (range 2-51). The median hemoglobin level increased by 2.0 gram/dL in responders. Eleven of 38 red blood cell (RBC) transfusion-dependent patients (29%) had clinically significant RBC transfusion reductions (reduction of 4 U/8 weeks, IWG 2006) with 4 patients (11%) achieving RBC transfusion independence and 3 patients continuing on treatment. In addition, one patient continued in complete remission for more than 12 months following discontinuation of therapy (Quddus, et. al., J. Hem. and Onc. April 2010, 3:15).

Telintra® continues to demonstrate multilineage hematologic improvement. There was a 15% Hematologic Improvement-Neutrophil (HI-N) rate observed in 3 of 20 patients (95% CI, 3.2-37.9), and the bilineage HI rate (HI-E and HI-N) was 11%, 2 of 19 patients (95% CI, 1.3-33.1).

There were three cytogenetic complete responses, one in a patient with 45X, −Y[4], 46, XY [16] abnormal cytogenetics that converted to normal after four cycles of therapy. Of the four patients enrolled in the study with del 5 q minus, two had a complete cytogenetic response, including one who had failed prior Revlimid® therapy.

A planned logistic regression analysis was used to evaluate all known prognostic characteristics in order to define those patients who had an increased likelihood of HI-E response to Telintra®. Prior DMTI treatment predicts a five-fold decrease in the odds for a HI-E response to Telintra® (p=0.023). Prior Revlimid® treatment was observed to enhance HI-E response to Telintra®.
There was a 40% HI-E rate (6 of 15 patients, 95% CI, 16.3%-67.7%) in patients who had prior Revlimid® treatment, but no prior DMTI treatment. Within this patient group, five of 11 patients (45%) achieved significant RBC transfusion reduction with three of those patients (27%) achieving transfusion independence.
There was a 26% HI-E rate (6 of 23 patients, 95% CI, 10.2%-48.4%) in patients who had no prior Revlimid® treatment and no prior DMTI treatment. Within this group, five of 11 patients (45%) achieved significant RBC transfusion reduction.
There was a 0% HI-E rate (0 of 17 patients, 95% CI, 0%-19.5%) in patients who had no prior Revlimid® treatment but who had received prior DMTI treatment.

More than 403 cycles of Telintra® therapy have been administered. The safety data is based on all patients treated. The most common non-hematologic adverse events (AEs) were Grade 1 and 2 gastrointestinal (GI) respectively, nausea (45%, 16%), diarrhea (25%, 7%) and vomiting (30%, 12%). Grade 3 events were uncommon: nausea (1%), diarrhea (3%) and vomiting (2%). Prior DMTI treatment was associated with an increased incidence of GI AEs.

Telintra® treatment may result in clinically significant hematologic improvement in patients with MDS and may offer an alternative to RBC transfusions. These results are consistent with levels of efficacy observed in prior studies with Telintra®, the first GST P1-1 enzyme inhibitor tested in MDS patients.

Tables 1 and 2 summarize the results of this clinical study.

TABLE 1
Hematological Improvement-Erythroid (HI-E): Time to Response
and Duration of Response Starting Telintra ® Dose
of 3,000 mg/day (1.5 g b.i.d.) or 2,000 mg/day (1 g b.i.d.)
(Efficacy Evaluable Population)

| Telintra ® Dosing Schedule | |
|---|---|
| 1.5 g b.i.d. 2 weeks on | 1 g b.i.d. 3 weeks on |
| & 1 week off | & 1 week off |
| (N = 29) | (N = 31) |

Time to HI-E Response (Weeks) [1]

| | | |
|---|---|---|
| N | 7 | 6 |
| Mean | 8.4 (0.72) | 8.9 (1.29) |
| Median | 8.1 | 8.4 |
| Min, Max | 8.0, 10.0 | 8.0, 11.3 |

Duration of HI-E Response (Weeks) [2]

| | | |
|---|---|---|
| # Event | 5 (71.4%) | 2 (33.3%) |
| # Censored | 2 (28.6%) | 4 (66.7%) |
| Median (95% CI) | 18.4 (3.1-51.0) | 46.1 (10.0-46.1) |
| Min, Max | 1.9-51.0 | 2.4-46.1 |

[1] Days from date of first dose of study medication to the date of first documentation of response plus one divided by 7.
[2] Total number of days of where response is seen divided by 7.

TABLE 2
Hematological Improvement - Erythroid (HI-E): by Revlimid ® and
DNA methyltransferase inhibitors failure status
(Efficacy evaluable population)

| | | HI-E [1] Statistics | | |
|---|---|---|---|---|
| | | | Response | Response Rate |
| Revlimid ® | DMTI [2] | N | (n) | (95% Confidence Interval) |
| Yes | Yes | 15 | 3 | 20% (4.3%-48.1%) |
| No | Yes | 15 | 0 | 0.0% (0.0%-21.8%) |
| No | No | 27 | 6 | 22.2% (8.6%-42.3%) |

[1] RBC transfusion reduction from baseline => 4 units per eight weeks; or patient with symptomatic anemia not transfusion dependent with hemoglobin <11 g/dL prior to treatment, achieving a hemoglobin increase by >= 1.5 g/dL sustained for a period of eight weeks.
[2] Including azacitidine or decitabine.

Table 2 shows that: (1) when Telintra® was given to patients with no prior treatment of either Revlimid® or a DMTI, the response rate to Telintra® was about 22%; (2) when Telintra® was given to patients with prior DMTI treatment, none of the patients responded to Telintra®; and (3) the response rate to Telintra® was about 20% for patients who were treated with both Revlimid® and a DMTI prior to treatment with Telintra®.

1. A method for treating a myelodysplastic syndrome in a patient who has been treated with a DNA methyltransferase inhibitor, which method comprises administering to said patient lenalidomide prior to and/or concurrently with administration of ezatiostat or a salt thereof.

2. The method of claim 1, wherein the lenalidomide is administered prior to administration of the ezatiostat or salt thereof.

3. The method of claim 1, wherein the lenalidomide is administered concurrently with administration of the ezatiostat or salt thereof.

4. The method of claim 1, wherein the ezatiostat or salt thereof is administered daily for at least 2 weeks.

5. The method of claim 1, wherein the ezatiostat or salt thereof is administered orally.

6. The method of claim 1, wherein the ezatiostat or salt thereof is ezatiostat hydrochloride.

7. The method of claim 6, wherein the ezatiostat hydrochloride is administered according to a treatment schedule comprising administration of a daily dosage of 2 grams for three weeks followed by a one-week interruption wherein no ezatiostat or a salt thereof is administered.

8. The method of claim 6, wherein the ezatiostat hydrochloride is administered accordingly to a treatment schedule comprising administration of a daily dosage of 3 grams for two weeks followed by a one-week interruption wherein no ezatiostat or a salt thereof is administered.

9. A composition for treating a myelodysplastic syndrome in a patient who has been treated with a DNA methyltransferase inhibitor, which composition comprises lenalidomide, ezatiostat or a salt thereof, and optionally a pharmaceutically acceptable excipient.

10. The composition of claim 9, wherein the ezatiostat or the salt thereof and the lenalidomide together are in a therapeutically effective amount.

11. A kit for treating a myelodysplastic syndrome in a patient who has been treated with a DNA methyltransferase inhibitor, which kit comprises a first composition comprising enalidomide and a second composition comprising ezatiostat or a salt thereof.

12. The composition of claim 11, wherein the ezatiostat or the salt thereof and the lenalidomide together are in a therapeutically effective amount.
